(19) United States
(12) Patent Application Publication
(10) Pub. No.: US 2004/0082783 A1
(43) Pub. Date: Apr. 29, 2004

(54) Bis (amino acid) molecular scaffolds

(76) Inventor: Christian E. Schafmeister, Pittsburgh, PA (US)

Correspondence Address: ECKERT SEAMANS CHERIN & MELLOTT, 600 GRANT STREET, 44TH FLOOR, PITTSBURGH, PA, 15219

(21) Appl. No.: 10/612,098

(22) Filed: Jul. 2, 2003

Related U.S. Application Data
(60) Provisional application No. 60/401,474, filed on Aug. 6, 2002.

(51) Int. Cl. C07D 487/14

(52) U.S. Cl. 544/231; 548/536

(57) ABSTRACT

The present invention provides molecular building blocks of rigid bis(amino acids). The molecular building blocks can be linked together through the formation of rigid diketopiperazine rings, to provide oligomers having the desired three dimensional structure. Oligomers formed from the basic building blocks are also disclosed.

### CROSS-REFERENCE TO RELATED APPLICATIONS

This application claims priority under 35 USC 199(e) to provisional application Serial No. 60/401,474, filed Aug. 6, 2002.

### FIELD OF THE INVENTION

The present invention provides molecular building blocks of rigid bis(amino acids). The molecular building blocks can be linked together through the formation of rigid diketopiperazine rings, to provide the desired three dimensional structure.

### BACKGROUND INFORMATION

In the size range between one nanometer and one hundred nanometers biology constructs an almost endless assortment of biological machines called proteins. They are the most basic functional components of life. They are the molecular machines that catalyze the chemical reactions, process the information, transduce energy between chemical/mechanical/electronic/photonic forms and serve as the structural scaffolding that makes life possible (FIG. 1). There is currently have no systematic way of constructing devices on this size scale, but it would be highly desirable because devices on this scale, theoretically, would be the most efficient for almost any process. This is one of the goals of the new field of nanotechnology.

### SUMMARY OF THE INVENTION

The present invention allows the systematic construction of molecular devices that approach biological proteins in terms of their capabilities and will have very broad application. Use of the molecular building blocks may lead to new sensors, chemical catalysts and components for molecular electronics, and to the development of molecular electronics based computers and microscopic machines that could swarm within the human body and destroy cancers under a doctors control.

The present invention allows the systematic construction of molecular devices in the size range between one nanometer and twenty-five nanometers. The present invention provides novel chemical compounds called molecular building blocks and the syntheses that are used to construct them from commercially available materials. It involves a novel synthetic process by which the building blocks are assembled into complex three-dimensional shapes that act as scaffolds to present functional groups.

The applications of this basic technology may be almost endless. As it becomes available to the larger scientific community, it may serve as a platform for many valuable applications. The commercial products could initially be the building blocks themselves, which could be sold as fine chemicals for use by scientists to construct nanoscale devices. In the later stage, specific applications of molecular devices made from the molecular scaffolds will be the commercial products.

### BRIEF DESCRIPTION OF THE DRAWINGS

The invention is further illustrated by the following non-limited drawings in which:

FIG. 1 is an illustration contrasting the construction of macromolecules via the biological polymer folding approach with the building block ladder oligomer approach of the present invention. On the left, the protein folding process, which is poorly understood, folds polypeptide chains into functional structures. On the right, the circles, triangles, squares represent rigid molecular building blocks that can be coupled through pairs of bonds in different sequences to construct different rigid shapes. Each sequence forms a specific, complex and rigid shape without involving an folding process.

FIG. 2: The structures of initial classes of molecular building blocks. Each contains two protected amino acid moieties that will be used to join the building blocks through rigid diketopiperazine rings. Each class is made of several stereoisomers accessed synthetically by controlling the stereochemistry at each stereocenter (labeled with a “*”).

FIG. 3: The synthetic route to the “proline” class of building blocks. This class consists of four stereoisomers of which all four are easily accessible.

FIG. 4: The steps required for the sequential formation of a rigid diketopiperazine linkage between two building blocks: (A) the protecting group “P” (i.e.: Boc) from the leading edge amine of building block “i” is removed. (B) an amide bond is formed by introducing the next building block “i+1” carrying an activated carboxylate derivative (i.e., acid fluoride). (C) the orthogonal protecting group “Q” is removed (i.e.: ortho-nitrobenzene sulfonyl). (D) the free amine spontaneously attacks the adjacent methyl ester to form a diketopiperazine ring. This approach can be used to synthesize arbitrary length rigid ladder oligomers.

FIG. 5: Formation of the diketopiperazine linkages in parallel. After cleavage of the oligomer from the solid support, reductive removal of the benzyloxycarbonyl (Cbz) groups followed by incubation of the oligomer in 20% piperidine in dimethyl-formamide causes closure of all of the diketopiperazine rings in parallel and formation of the ladder oligomer.

FIG. 6: This diagram illustrates the concept of cavity containing scaffolds displaying a bound metalloporphyrin that could serve as colorimetric ligand sensors. Different scaffolds will have varying selectivity for ligands that can access the bound metalloporphyrin.

### DETAILED DESCRIPTION OF PREFERRED EMBODIMENTS

The molecular building blocks are novel small molecules that are synthesized from commercially available starting materials using syntheses that we have developed (FIG. 2). They are grouped into several classes, each class containing several stereoisomers. The synthetic procedures for constructing the building blocks must be short and economical; ideally, they involve less than ten steps.

In one aspect, the present invention provides a compound having the formula:

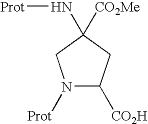

The CO₂Me, CO₂H, and NH-Prot groups are attached to the central pyrrole ring via cis- or trans-bonding. Compounds of the above formula can have the moieties attached at the stereocenters in any combination of configuration (R) and (S), i.e., CO₂H and NH-Prot can both be in cis, trans or oppositely configured.

As used herein the term “protecting group” (and abbreviated as “prot”) refers to a moiety that protects the atom of interest from attack during synthesis, and which can be easily removed at a later stage during formation of the desired compound of interest. Protecting groups are well known in the art. Suitable protecting groups include, but are not limited to, Boc, Ns, Fmoc and Cbz as defined by the following formulas:

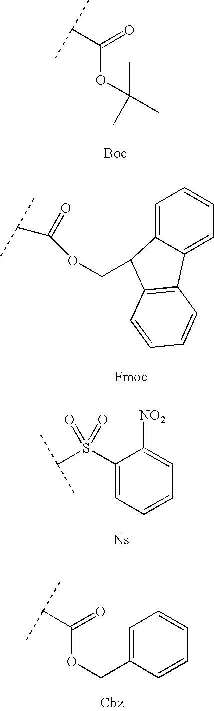

The first class of molecular building blocks, the “proline” monomer class, has been synthesized as shown in (Scheme 1). The synthesis starts from the inexpensive chiral starting material trans-4-hydroxy-L-proline and uses a key Bucherer-Bergs reaction[1] to convert a ketone into an amino acid through a hydantoin. These building blocks display two differentially protected α-amino acids on a five membered ring. They hold their preceding and following partners in an extended orientation and can be combined to form extended rods. The distance from one monomer to the next in an oligomer is about 5 Å, allowing us to construct molecular rods with defined lengths of 5 Å, 10 Å, 15 Å, 20 Å etc. An oligomer containing 20 proline building blocks would form a rod 100 Å long. The flexibility of this rod can be determined experimentally. The five membered ring of the proline building block may flip between different envelope conformations imparting some flexibility into the polymer.

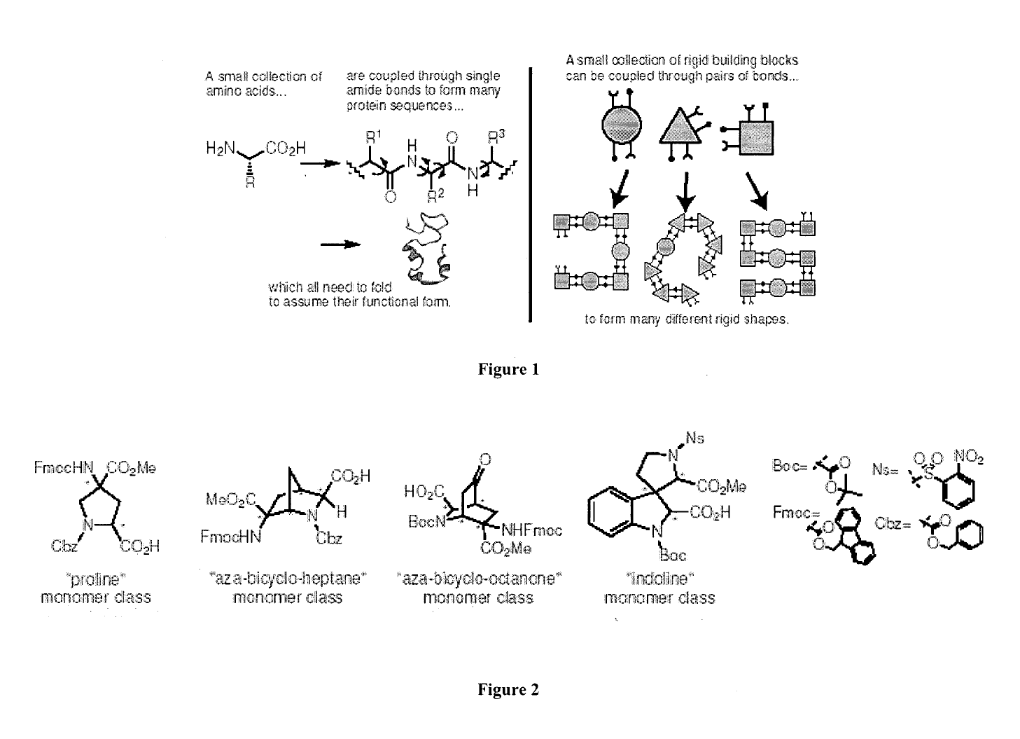

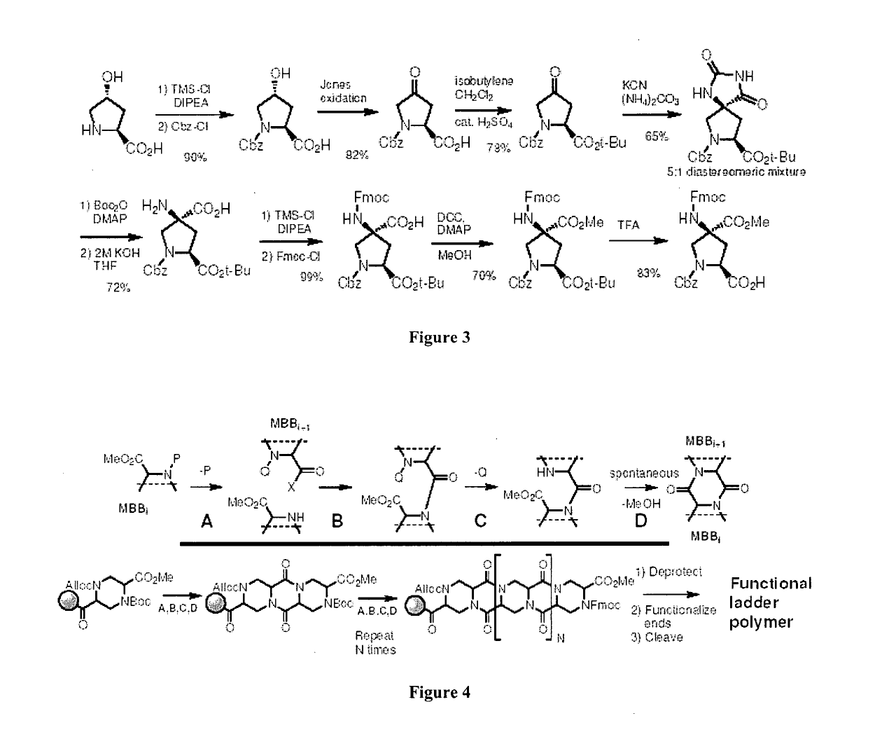

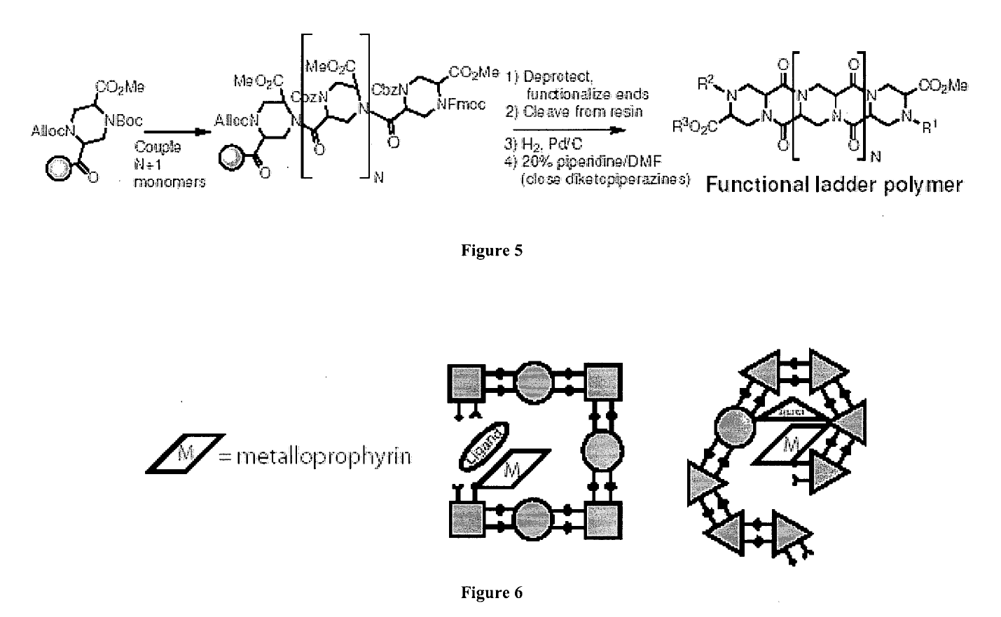

The enantiomer of this building block can be synthesized by starting from the same trans-4-hydroxy-L-proline starting material through epimerization of the α-carbon to form cis-4-hydroxy-D-proline using a known procedure [2]. The building blocks are rigid bis(amino acids) and the unique approach involves coupling them through pairs of amide bonds (FIG. 4, FIG. 5) to form sequence specific rigid ladder oligomers on solid support. Each building block has a unique rigid three-dimensional structure. When these are assembled in different sequences they form ladder oligomers with specific three-dimensional shapes (FIG. 1).

In an additional aspect, the present invention provides oligomers formed from the “proline” monomer building block described above, the oligomer having the formula:

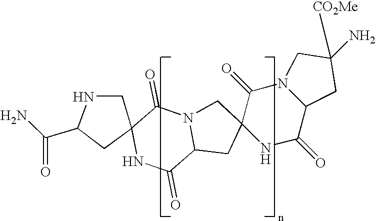

where the configuration of the terminal CO₂Me, NH₂, and CONH₂ groups are (R) or (S). The configuration of all of the stereocenters is defined by the configuration of the monomers that make the oligomer and can be any combination of (R) and (S). The subscript “n” can be any number less than about 100, e.g., 1, 2, 3, 5, 10, 15, 20, 30, 40, 50, 75, and the like.

Thus, the power of synthetic organic chemistry to make small asymmetric molecules is combined with the power of polymer synthesis to allow rapid assembly of macromolecules. Solid phase peptide synthesis has made it possible to synthesize peptides in excess of 50 amino acids in length with excellent yields[3]. Synthesis of molecules with defined shapes in the range of 1,000 to 10,000 Daltons is possible. Using the approach of the present invention, using just four building blocks and assembling sequences of ten monomers, 4¹⁰ or about 1,000,000 different rigid macromolecular shapes can be constructed. The synthesis of every one of these million different molecules is quick and follows exactly the same synthetic steps (but using different building blocks) on solid support. The combination of rapid design and synthesis will enable a short development cycle for molecular devices. The applications of this technology are almost endless. Once the monomers are commercially available and the force field and software tools have been developed, it could be used by any chemist to design and synthesize functional macromolecules, such as catalysts, sensors and nano-scale molecular devices.

One conceptual application involves the development of specific colorimetric sensors. Array based vapor-sensing devices have been developed that utilize arrays of metalloporphyrin dyes to detect ligand binding [5]. By virtue of a strong color change, the devices register ligands binding to metalloporphyrins containing Sn⁴⁺, Co³⁺,Cr³⁺, Mn³⁺,Fe³⁺,Co²⁺,Cu²⁺,Ru²⁺,Zn²⁺ and Ag²⁺. The metalloporphyrins in these devices are spotted onto reverse phase silica plates and show excellent stability within the device. These devices are able to detect strong ligands such as alcohols, amines, ethers, phosphines, phosphites, thioesters and thiols as well as weakly ligating arenes, alocarbons and ketones. At the simplest level this device is a square grid of dots that change color based on the odorant that is impinging on the device. The odorant could be identified by eye using a collection of calibrated color charts for comparison or the color grid could be read electronically and identified by a computer. The simple metalloporphyrins that have been used to date are able to distinguish between compounds where the nature of the coordinating atom is different (amines vs. alcohols or phosphines vs. phosphites). However, it stands to reason that they would be less sensitive to the (more interesting) structure of groups attached to the coordinating atom and completely insensitive to their stereochemical nature. By synthesizing ladder oligomers that form chiral cavities encapsulating covalently attached met-alloporphyrins, a large collection of highly selective colorimetric sensors could be constructed. The shape of the cavity and its stereochemistry could distinguish structural and stereochemical features of the ligand that are very far from the coordinating atom. In principle, sensors that distinguish between subtly different ligands like (S)-(−)-propylene oxide and (R)-(+)-propylene oxide could also be constructed. Monomer sequences that form cavities would be found using computer searches or rational design. It would be easy to identify unsuccessful sensors visually by their lack of reaction to very small ligands, or by their lack of discrimination between more complex ligands. Once successful sensors have been developed, the structural basis of their selectivity through X-ray crystallography could be determined.

The most toxic and odiferous compounds tend to be excellent ligands for metal ions [5] and may irreversibly bind to an unhindered metalloporphyrins. A sensor would be of greater value if it could reversibly bind strongly coordinating ligands. Using the X-ray crystal structure of such a strong ligand irreversibly bound to one of our scaffold based sensors, we could re-engineer the cavity to position a sterically bulky group over the metal center and weaken the binding of the ligand without eliminating it. This is analogous to the model in which the distal histidine in hemoglobin lowers the affinity of the bound heme for carbon monoxide relative to oxygen.

Specific scaffold/metalloporphyrin based sensors could be used in many applications. They could detect chemical warfare agents, spoiled food and industrial wastes in real time. This sensor technology could ultimately be integrated into a device that acts like a very sensitive “electronic nose”; able to identify an enormous number of volatile compounds in real time from complex mixtures.

Whereas particular embodiments of this invention have been described above for purposes of illustration, it will be evident to those skilled in the art that numerous variations of the details of the present invention may be made without departing from the invention as defined in the appending claims.

Literature Cited

References

[1] Asymmetric syntheses of all four isomers of 4-amino-4-carboxyproline: Novel conformationally restricted glutamic acid analogues. K. Tanaka and H. Sawanisi. Tetrahedron: Asymmetry, 6(7):1641-1656, 1995.

[2] Transition-metal-catalyzed asymmetric organic-synthesis via polymer-attached optically-active phosphine-ligands .5. preparation of amino-acids in high optical yield via catalytic-hydrogenation. G. L. Baker, S. J. Fritschel, J. R. Stille, and J. K. Stille. Journal of Organic Chemistry, 46(14):2954-2960, 1981.

[3] Constructing proteins by dovetailing unprotected synthetic peptides -backbone-engineered hiv protease. M. Schnolzer and S. B. H. Kent. Science, 256(5054):221-225, 1992.

[4] A 2nd generation forcefield for the simulation of proteins, nucleic-acids, and organic-molecules. W. D. Cornell, P. Cieplak, C. I. Bayly, I. R. Gould, K. M. Merz, D. M. Ferguson, D. C. Spellmeyer, T. Fox, J. W. Caldwell, and P. A. Kollman. Journal of the American Chemical Society, 117(19):5179-5197, 1995.

[5] A colorimetric sensor array for odour visualization. N. A. Rakow and K. Suslick. Nature, 406:710{713, 2000.

What is claimed is:

1. A compound having the formula: 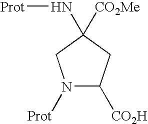
wherein the CO₂Me, CO₂H, and NH-Prot groups are attached to the central pyrrole ring via cis- or trans-bonding.

2. The compound of claim 1, wherein the protecting group is selected from the group consisting of Boc, Ns, Fmoc and Cbz.

3. The compound of claim 1, wherein CO₂H has the configuration (S) and the quaternary center has the configuration (S).

4. The compound of claim 1, wherein CO₂H has the configuration (S) and the quaternary center has the configuration (R).

5. The compound of claim 1, wherein CO₂H has the configuration (R) and the quaternary center has the configuration (S).

6. The compound of claim 1, wherein CO₂H is has the configuration (R) and the quaternary center has the configuration (R).

7. A compound having the formula: 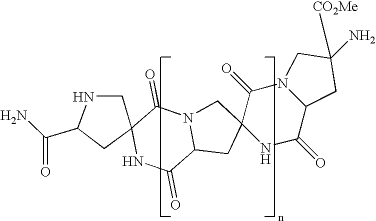
wherein n is less than about 100 and the configurations of all stereocenters are defined by the monomers that went into making the oligomer and can be any combination of (R) and (S).

8. The compound of claim 8, wherein n is less than 50.

9. The compound of claim 8, wherein n is less than 40.

10. The compound of claim 8, wherein n is less than 30.

11. The compound of claim 8, wherein n is less than 20.

12. The compound of claim 8, wherein n is less than 20.
